(19) United States
(12) Patent Application Publication
(10) Pub. No.: US 2014/0135700 A1
(43) Pub. Date: May 15, 2014

(54) SYSTEMS AND METHODS FOR DELIVERING A THERAPEUTIC AGENT USING A CLAMPED ACTUATOR

(71) Applicant: SpringLeaf Therapeutics, Inc., Boston, MA (US)

(72) Inventor: J. Richard Gyory, Sudbury, MA (US)

(73) Assignee: SpringLeaf Therapeutics, Inc., Boston, MA (US)

(21) Appl. No.: 13/673,470

(22) Filed: Nov. 9, 2012

(51) Int. Cl. A61M 5/145

(52) U.S. Cl. CPC A61M 5/14586
USPC 604/134; 604/151

(57) ABSTRACT

A delivery system includes a reservoir, a fluid communicator, and an actuator. The reservoir is configured to contain a fluid and is in fluid communication with the fluid communicator. The actuator includes an unconstrained first end portion, an unconstrained second end portion, and a constrained medial portion therebetween. When the actuator is actuated the medial portion is configured to bend along a bend axis to produce a displacement of the first end portion and the second end portion relative to the medial portion. The bending of the actuator is configured to displace the actuator towards the reservoir to exert a force on the reservoir such that a fluid disposed within the reservoir is communicated through the fluid communicator.

### BACKGROUND

Embodiments described herein relate generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid some of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Furthermore, modulating or controlling the delivery of drugs using such systems may not be possible without using complex systems.

Some infusion pump systems may be large and may require tubing between the pump and the infusion set, which can impact the quality of life of the patient. Moreover, some infusion pumps can be expensive and may not be disposable. As such, a need exists for improved systems and methods for delivering a therapeutic agent into a body.

### SUMMARY

Devices and methods for delivering a therapeutic agent to a patient are disclosed herein. In some embodiments, a delivery system includes a reservoir, a fluid communicator, and an actuator. The reservoir is configured to contain a fluid and is in fluid communication with the fluid communicator. The actuator includes an unconstrained first end portion, an unconstrained second end portion, and a constrained medial portion therebetween. When the actuator is actuated, the medial portion is configured to bend along a bend axis to produce a displacement of the first end portion and the second end portion relative to the medial portion. The bending of the actuator is configured to displace the actuator towards the reservoir to exert a force on the reservoir such that a fluid disposed within the reservoir is communicated through the fluid communicator.

### BRIEF DESCRIPTION OF THE DRAWINGS

FIG. 1 is a schematic illustration of a delivery system according to an embodiment.

FIGS. 2A and 2B are schematic illustrations of an actuator assembly shown in a first configuration and a second configuration, respectively, according to an embodiment.

FIGS. 3A and 3B are schematic illustrations of an actuator assembly shown in a first configuration and a second configuration, respectively, according to an embodiment.

FIGS. 4A and 4B are schematic illustrations of an actuator assembly shown in a first configuration and a second configuration, respectively, according to an embodiment.

FIGS. 5A and 5B are schematic illustrations of an actuator assembly shown in a first configuration and a second configuration, respectively, according to an embodiment.

FIGS. 6A and 6B are schematic illustrations of an actuator assembly shown in a first configuration and a second configuration, respectively, according to an embodiment.

FIG. 7 is a perspective view of a delivery system according to an embodiment.

FIG. 8 is an exploded view of the delivery system of FIG. 7.

FIG. 9 is a perspective view of an actuator assembly included in the delivery device of FIG. 7, in a first configuration.

FIG. 10 is a cross-sectional view of the actuator assembly of FIG. 9 in its first configuration, taken along the line 10-10 in FIG. 9.

FIG. 11 is a perspective view of the actuator assembly of FIG. 9 in a second configuration.

FIG. 12 is a cross-sectional view of the actuator assembly of FIG. 9 in its second configuration, taken along the line 12-12 in FIG. 11.

### DETAILED DESCRIPTION

Devices and methods for delivering a therapeutic agent to a patient are disclosed herein. In some embodiments, a delivery device includes a reservoir, a fluid communicator, and an actuator. The reservoir is configured to contain a fluid and is in fluid communication with the fluid communicator. The actuator includes an unconstrained first end portion, an unconstrained second end portion, and a constrained medial portion therebetween. When the actuator is actuated, the medial portion is configured to bend along a bend axis to produce a displacement of the first end portion and the second end portion relative to the medial portion. The bending of the actuator is configured to displace a portion of the actuator towards the reservoir to exert a force on the reservoir such that a fluid disposed within the reservoir is communicated through the fluid communicator.

In some embodiments, a delivery device includes a reservoir, a first actuator, a second actuator, and a constraining member configured to couple a portion of the first actuator to a portion of the second actuator. The first actuator is movable between a first configuration and a second configuration such that the first actuator exerts a first force on the reservoir when the first actuator is moved from its first configuration to its second configuration to urge fluid within the reservoir out of the reservoir. The second actuator is movable between a first configuration and a second configuration such that the second actuator exerts a second force on the reservoir when the second actuator is moved from its first configuration to its second configuration to urge fluid within the reservoir out of the reservoir. The first actuator further defines a first stroke when the first actuator is moved from its first configuration to its second configuration. The second actuator further defines a second stroke when the second actuator is moved from its first configuration to its second configuration. The first stroke and the second stroke collectively define a stroke of the delivery device.

In some embodiments, a delivery device includes a reservoir, a first electrochemical actuator, and a second mechanical actuator. The first actuator is movable between a first configuration in which the first actuator is substantially planar and a second configuration in which at least a portion of the first actuator is moved substantially perpendicular to the plane of its first configuration. The first actuator is configured to exert a first force on the reservoir when the first actuator is moved from its first configuration to its second configuration to urge fluid within the reservoir out of the reservoir. The second actuator is movable between a first configuration and a second configuration such that the second actuator exerts a second force on the reservoir when the second actuator is moved from its first configuration to its second configuration to urge fluid within the reservoir out of the reservoir. The first actuator is configured to maintain the second actuator in its first configuration when the first actuator is in its first configuration.

Devices, systems and methods described herein are configured for use in the delivery of therapeutic agents to a patient's body. Such therapeutic agents can be, for example, one or more drugs and can be in a fluid form of various viscosities. In some embodiments, the devices and methods can include a delivery device that includes an actuator, such as, for example, an electrochemical actuator, which can have characteristics of both a battery and a pump. Specifically, an electrochemical actuator can include an electrochemical cell that produces a pumping force as the cell discharges. Thus, the delivery device can have relatively fewer parts than conventional drug pumps and can be more compact and/or more reliable than conventional drug pumps as well as being disposable. Such drug delivery devices can be desirable, for example, for use in delivery devices that are designed to be attached to a patient's body (e.g., a wearable device). Therefore, the attributes of the delivery device may reduce the cost and may reduce the discomfort associated with infusion drug therapy.

The devices, systems and methods described herein can include an electrochemical actuator, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Pat. No. 7,872,396, entitled “Electrochemical Actuator” by Chiang et al., U.S. Pat. No. 7,999,436, entitled “Electrochemical Actuator” by Chiang et al., U.S. Pat. No. 7,828,771, entitled “Systems and Methods for Delivering Drugs” by Chiang et al., (the '771 patent), and U.S. Pat. No. 8,247,946, entitled “Electrochemical Actuator” by Chiang et al., (collectively referred to herein as the “the Electrochemical Actuator applications”), the disclosures of which are incorporated herein by reference in their entirety. Such electrochemical actuators can include at least one component that responds to the application of a voltage or current by experiencing a change in volume or position. The change in volume or position can produce mechanical work configured to act on a fluid source (e.g., fluid reservoir 180 described below) or may be transferred to a fluid source, such that a fluid can be delivered out of the fluid source to an insertion mechanism for delivery to a patient.

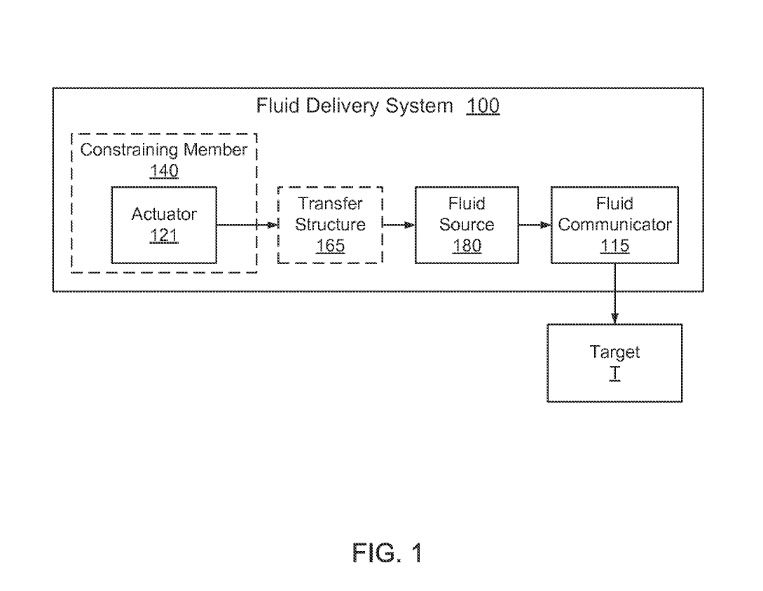

FIG. 1 is a schematic block diagram of a fluid delivery system 100 (also referred to herein as “delivery device” or “drug delivery device”), according to an embodiment. The fluid delivery system 100 includes at least an actuator 121, a fluid source 180, and a fluid communicator 115. The delivery device 100 can contain a fluid (i.e., a therapeutic agent) to be delivered into a target T (e.g., a human or other mammalian body in need of a drug therapy or prophylaxis) via the fluid communicator 115, as further described herein.

The actuator 121 of the delivery device 100 can be any suitable actuator 121 that can actuate or otherwise create a pumping (e.g., driving) force. The actuator 121 can be movable between a first configuration and a second configuration. In some embodiments, the actuator 121 can be a device that experiences a change in volume, length, area, or position in response to an activation event (e.g., a mechanical and/or an electrical activation). For example, in some embodiments, the actuator 121 can be an electrochemical actuator configured to experience a volumetric change in response to an electrochemical reaction that occurs therein. Expanding further, the actuator 121 can be an electrochemical actuator that includes a charged electrochemical cell, and at least a portion of the electrochemical cell can actuate as the electrochemical cell discharges. Thus, the actuator 121 can be considered a self-powered actuator or a combination battery and actuator.

In some embodiments, the actuator 121 can be a mechanical actuator such as, for example, a spring or the like. For example, in some embodiments, the actuator 121 can be a leaf spring, a linear spring, a compression spring, a torsion spring, a Belleville spring, or the like. In such embodiments, the actuator 121 (e.g., spring) can have any suitable kinetic potential such that when the actuator 121 is moved from the first configuration (associated with a relatively high kinetic potential) to the second configuration (associated with a relatively low kinetic potential), the actuator 121 exerts a driving force.

In some embodiments, the delivery system 100 can include more than one actuator 121. In such embodiments, the actuators 121 can be similar or dissimilar actuators 121. For example, in some embodiments, the delivery system 100 can include two or more electrochemical actuators of similar configuration. In other embodiments, the delivery system can include two or more electrochemical actuators having differing electrochemical potential. In still other embodiments, the delivery system 100 can include one or more electrochemical actuator and one or more mechanical actuator. In such embodiments, the mechanical actuator(s) and the electrochemical actuator(s) can collectively exert a driving force that can be greater than a driving force produced by mechanical actuator or an electrochemical actuator alone.

While not shown in FIG. 1, the actuator 121 can include a first end portion, a second end portion, and a medial portion disposed therebetween. In some embodiments, the delivery system 100 can be configured to constrain the actuator 121 at the medial portion while the first end portion and the second end portion remain unconstrained. For example, in some embodiments, the delivery system 100 can optionally include a constraining member 140 that constrains the medial portion. In some embodiments, the actuator 121 can be configured to deflect or bend when activated such that the actuator 121 bends in or at the medial portion along a bend axis. In this manner, the first end portion and the second end portion (e.g., the unconstrained portions) can be displaced relative to the medial portion (e.g., the constrained portion) to move the actuator 121 to its second configuration. Furthermore, the displacement of the first end portion and the second end portion, relative to the medial portion, can be such that the actuator 121 exerts a force on the fluid source 180 to deliver the fluid from the fluid source 180 into the fluid communicator 115 as described in more detail below.

The fluid source 180 of the delivery device 100 can be any component capable of retaining a fluid or drug in fluid form. For example, the fluid source 180 can be a reservoir, a pouch, a chamber, a barrel, a bladder, or other known device that can contain a drug in fluid form therein. In some embodiments, the fluid source 180 may be disposable (e.g., not intended to be refillable or reusable). In other embodiments, the fluid source 180 can be refilled, which may permit reusing at least a portion of the device and/or varying the drug or fluid delivered by the device.

The fluid source 180 can have any suitable size or shape. In some embodiments, the size of the fluid source 180 can correspond to an electrochemical or kinetic potential of the actuator 121. For example, the size and/or volume of the fluid source 180 can be selected so that the fluid source 180 becomes substantially empty at about the same time that the actuator 121 becomes substantially actuated. By optimizing the size of the fluid source 180 and the amount of drug contained therein to correspond to the driving potential (e.g., the stroke) of the actuator 121, the size and/or cost of the device may be reduced. In other embodiments, the fluid source 180 can be undersized relative to the actuator 121, thereby ensuring full discharge of the fluid contained therein.

In some embodiments, the delivery system 100 can include more than one fluid source 180. In such embodiments, a single device can be configured to deliver two or more drugs or fluids. The two or more drugs or fluids can be delivered discretely, simultaneously, alternating, according to a program or schedule, or in any other suitable manner. Moreover, the fluid sources 180 can be associated with the same or different actuators 121, the same or different fluid communicators 115, the same or different operational electronics (not shown in FIG. 1), and/or the same or different portions of other components of the delivery system.

The fluid communicator 115 can be in, or can be moved into, fluid communication with the fluid source 180. The fluid communicator 115 can be, for example, a needle, catheter, cannula, infusion set, or other known drug delivery conduit defining a lumen that can be inserted into or otherwise associated with the target T for drug delivery. In some embodiments, the fluid communicator 115 can be included in an insertion assembly or mechanism (not shown in FIG. 1). In such embodiments, the activation of the insertion assembly can cause the fluid communicator 115 to place the fluid source 180 in fluid communication with the target T. Similarly stated, in some embodiments, the activation of the insertion assembly can be operative in moving the fluid communicator 115 relative to a patient such that a portion of the fluid communicator 115 pierces a target tissue site to be disposed within the body. Thus, the fluid communicator 115 can define a flow path (e.g., via the lumen) between the fluid source 180 and the target T.

In use, the delivery device 100 can be placed in contact with the target T (e.g. placed on the surface of a patient's body), such that the fluid communicator 115 (e.g., a needle, cannula, etc.) is disposed adjacent to a desired injection site. The fluid communicator 115 can be activated with the actuation of the actuator 121 or separately such that a portion of the fluid communicator 115 is inserted into the patient's body (example embodiments illustrating various configurations for actuation of the fluid communicator 115 are described in the '771 patent incorporated by reference above). In the same process or in a subsequent process, the actuator 121 can be actuated to apply a force on the fluid source 180, causing the fluid to be delivered through the fluid communicator 115 and into the target T. For example, in some embodiments, the actuator 121 (e.g., an electrochemical actuator) can be actuated such that one or more portions (e.g., a first end portion and a second end portion described above) are displaced to apply a force on the fluid source 180 to pump the fluid out of the fluid source 180, through the fluid communicator 115, and into the target T.

In some embodiments, the delivery device 100 can optionally include a transfer structure 165 disposed between the actuator 121 and the fluid source 180. In such embodiments, the actuator 121 can exert a force (e.g., as described above) to move the transfer structure 165 relative to the fluid source 180. In this manner, the transfer structure 165 can be configured to evenly distribute the force exerted by the actuator 121 along a surface of the fluid source 180. In other embodiments, the transfer structure 165 can be shaped or configured to selectively distribute the force exerted by the actuator 121 on the fluid source 180 (e.g., in a peristaltic, stepwise, or gradually increasing fashion). In this manner, the actuator 121 can exert a force on the transfer structure 165 which can in turn exert a force on the fluid source 180 to pump the fluid out of the fluid source 180, through the fluid communicator 115, and into the target T.

Unlike conventional drug pumps, external tubing to communicate fluid from a fluid reservoir into the body can be eliminated. Such tubing can instead be contained within the delivery device 100, and the fluid communicator 115 can extend from the delivery device 100 into the body. Once the actuator 121 has completely discharged or the fluid source 180 (e.g. reservoir) is empty, the delivery device 100 can be removed from contact with the body of the patient. In some embodiments, the delivery device 100 is sufficiently inexpensive such that the delivery device 100 can be discarded. The delivery device 100 can permit drug delivery, such as subcutaneous or intravenous drug delivery, over a time period that can vary from several minutes to several days. Subsequently, the delivery device 100 can be removed from the body and discarded.

While not shown in FIG. 1, the components of the delivery system 100 can be fixedly or releasably coupled to and/or disposed within a housing. The housing can be removably or releasably attached to the body (e.g., the skin) of the patient. In some embodiments, a surface of the housing can include a removable adhesive such that the delivery device 100 can be adhered to the skin of a patient. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. In other embodiments, the delivery device 100 need not include an adhesive and can be associated with the skin, or generally with the body, in any other manner such as with a strap or band.

The size, shape, and weight of the delivery device 100 can be selected so that the delivery device 100 can be comfortably worn on the skin after the device is placed in contact thereon (e.g., via the adhesive). For example, the delivery device 100 can have a size, for example, in the range of about 1.0″×1.0″×0.1″ to about 5.0″×5.0″×1.0″, and in some embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml, and in some cases in the range of about 0.3 ml to about 100 ml, such as between about 0.5 ml and about 5 ml. The shape of the delivery device can be selected so that the delivery device 100 can be relatively imperceptible under clothing. For example, the housing can be relatively smooth and free from sharp edges.

In some embodiments, the use of an electrochemical actuator (described above) can further reduce the size and weight of the delivery device 100 by acting as both the actuator 121 and a battery. For example, in some embodiments, an electrochemical actuator can be in a charged state prior to being actuated and can electrically discharge when actuated to both deform (as described above) and supply a flow of current for various electrical components.

In some embodiments, the fluid delivery system 100 can be used to deliver a drug formulation which comprises a drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympnic, intraaural, topical, epidural, and/or peri-neural depending on, for example, the location of the fluid communicator 115 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Electrochemical actuators can provide volume-efficient capabilities that are especially effective in applications where minimal weight and volume are desired. Example applications are those of drug/medication patch pumps that are worn by a patient. While most pumps use a variety of prime movers that either require external drive circuitry or power, bulky, expensive, and/or complex, electrochemical actuator-based pumps have significant advantages by virtue of having a small actuator volume and no need for an external power source.

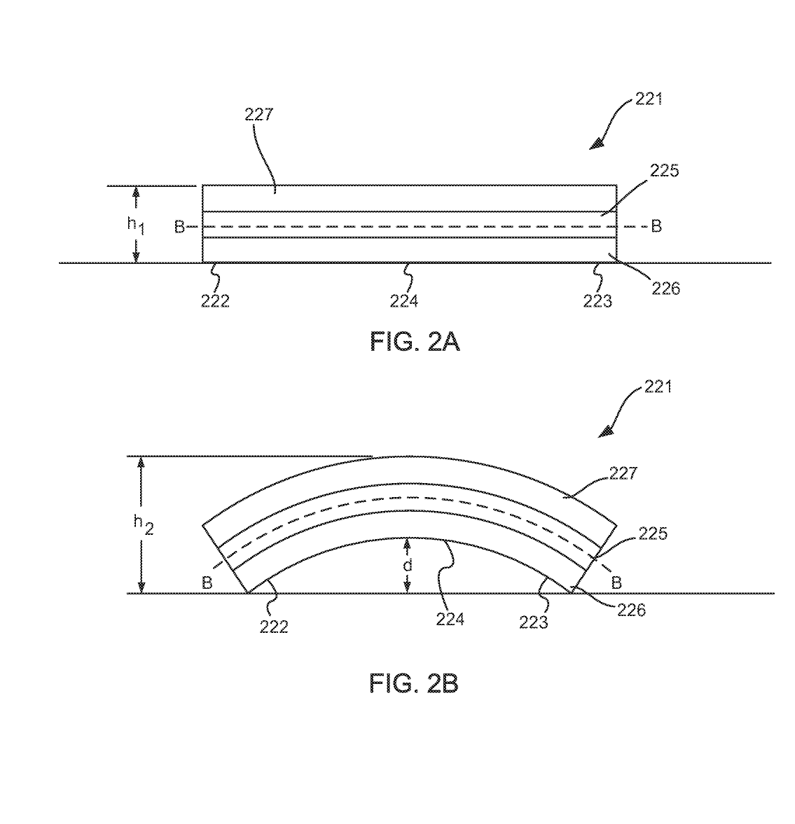

Referring now to FIGS. 2A and 2B, an electrochemical actuator 221 is illustrated, in a first configuration and a second configuration, respectively, according to an embodiment. The electrochemical actuator 221 (also referred to herein as “actuator”) can be an elongate plate including a first end portion 222, a second end portion 223, and a medial portion 224 disposed therebetween. The actuator 221 further includes a positive electrode 225, a negative electrode 226, and an electrolyte 227 that can form, for example, an electrochemical cell. The actuator 221 can be initially charged prior use such that the actuator 221 is substantially planar, as shown in FIG. 2A, and can be discharged during use such that the actuator 221 is deformed, as shown in FIG. 2B.

As shown in FIG. 2A, the actuator 221 has a first height h₁ when in its first configuration (e.g., a charged state prior to actuation). The positive electrode 225 can be configured to expand or displace in the presence of the electrolyte 227. For example, when a circuit between the positive electrode 225 and the negative electrode 226 is closed, current can travel from the positive electrode 225 to the negative electrode 226. The positive electrode 225 can then experience a change in volume or shape, resulting in a longitudinal displacement of at least a portion of the positive electrode 225. More specifically, the displacement of the positive electrode 225 can cause the actuator 221 to bend, buckle, fold, cup, elongate, contract, or otherwise experience a change in volume, size, shape, orientation, arrangement, or location, in or at the medial portion 224 along a bend axis B of the actuator 221.

Said another way, the first end portion 222, the second end portion 223, and the medial portion 224 can be substantially planar prior to the actuation of the actuator 221, and when the actuator 221 is discharged at least the medial portion 224 can displace (e.g., bend or flex) a non-zero distance d relative to the first end portion 222 and the second end portion 223. In this manner, the overall height h₁ of the actuator 221 can increase to a second height h₂ that is larger than first height h₁. Thus, the actuator 221 has a displacement or stroke that is equal to h₂-h₁. In some embodiments, the distance d is equal to the stroke length h₂-h₁. In other embodiments, the volume of the medial portion 224 can change (e.g., increase or decrease) when the medial portion 224 is deformed. Therefore, in some embodiments, the distance d need not be equal to the stroke length (e.g., can be greater than or less than the stroke length). As the actuator 221 is displaced, the actuator 221 can exert a pumping force or pressure on a fluid reservoir (not shown) and/or an associated transfer structure (not shown) coupled thereto. The pumping force or pressure exerted by the actuator 221 can cause a volume of fluid (e.g., a therapeutic agent) to be pumped out of the fluid reservoir. Thus, the electrochemical actuator 221 can be considered a self-powered electrochemical pump.

In this embodiment, the electrochemical actuator 221 has a positive electrode 210 selected to have a lower chemical potential for the working ion when the electrochemical actuator 221 is charged, and is thereby able to spontaneously accept working ions from the negative electrode 212 as the actuator is discharged. In some embodiments, the working ion can include, but is not limited to, the proton or lithium ion. When the working ion is lithium, the positive electrode 210 can include one or more lithium metal oxides including, for example, LiCoO₂, LiFePO₄, LiNiO₂, LiMn₂O₄, LiMnO₂, LiMnPO₄, Li₄Ti₅O₁₂, and their modified compositions and solid solutions; oxide compound comprising one or more of titanium oxide, manganese oxide, vanadium oxide, tin oxide, antimony oxide, cobalt oxide, nickel oxide or iron oxide; metal sulfides comprising one or more of TiSi₂, MoSi₂, WSi₂, and their modified compositions and solid solutions; a metal, metal alloy, or intermetallic compound comprising one or more of aluminum, silver, gold, boron, bismuth, gallium, germanium, indium, lead, antimony, silicon, tin, or zinc; a lithium-metal alloy; or carbon comprising one or more of graphite, a carbon fiber structure, a glassy carbon structure, a highly oriented pyrolytic graphite, or a disordered carbon structure. The negative electrode 212 can include, for example, lithium metal, a lithium metal alloy, or any of the preceding compounds listed as positive electrode compounds, provided that such compounds when used as a negative electrode are paired with a positive electrode that is able to spontaneously accept lithium from the negative electrode when the actuator is charged. These are just some examples, as other configurations are also possible.

In some embodiments, the electrochemical actuator can include an anode, a cathode, and a species, such as a lithium ion. In some embodiments, a source of lithium ion is the electrolyte which is made up an organic solvent such as propylene carbonate (PC), gamma butyl lactone (GBL), dioxylane, and others, and an added electrolyte. Some example electrolytes include LiPF₆, LiBr, and LiBF₄. At least one of the electrodes can be an actuating electrode that includes a first portion and a second portion. The portions can have at least one differing characteristic, such that in the presence of a voltage or current, the first portion responds to the species in a different manner than the second portion. For example, the portions can be formed from different materials, or the portions can differ in thickness, dimension, porosity, density, or surface structure, among others. The electrodes can be charged, and when the circuit is closed, current can travel. The species can, intercalate, de-intercalate, alloy with, oxide, reduce, or plate with the first portion to a different extent than the second portion. Due to the first portion responding differently to the species than the second portion, the actuating electrode can experience a change in one or more dimensions, volume, shape, orientation, or position.

In some embodiments, an actuator can be clamped or otherwise constrained at a desired location along a bend axis of the actuator such that portions other than a constrained portion deflect relative to the constrained portion. Expanding further, selectively constraining a portion of the actuator can produce varying deflection characteristics of the unconstrained portions of the actuator during discharge. For example, in some embodiments, an actuator can be constrained at an end portion. In such an arrangement an increased range of motion at a free end (i.e., an unconstrained end), opposite the constrained end, for the same angular deflection of the electrochemical actuator can be achieved and/or an increased rate of actuation for the same vertical tip deflection. Example embodiments of delivery devices including electrochemical actuators constrained at an end portion are generally described in U.S. Patent Publication No. 2011/0275998, entitled “Systems and Methods for Delivering a Therapeutic Agent,” filed on May 6, 2011, the disclosure of each of which is incorporated herein by reference.

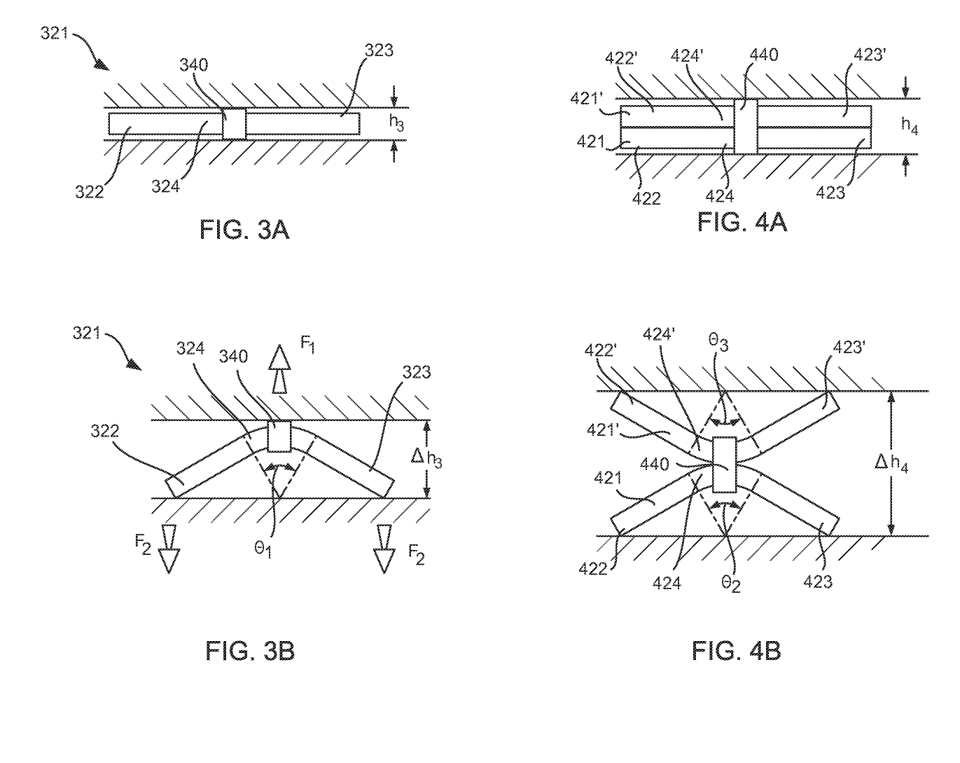

In other embodiments, an actuator can be constrained at a portion other than an end portion. For example, FIGS. 3A and 3B illustrate an electrochemical actuator 321 according to an embodiment, in a first configuration and a second configuration, respectively. The electrochemical actuator 321 (also referred to herein as “actuator”) includes a first end portion 322, a second end portion 323, and a medial portion 324 disposed therebetween. As shown in FIG. 3A, the actuator 321 can be substantially planar when in the first configuration (e.g., a charged configuration) and can have a height h₃. Furthermore, the actuator 321 can be arranged such that the medial portion 324 is constrained by a constraining member 340. The constraining member 340 can be any suitable member 340 configured to constrain the movement of a portion of the actuator 321. For example, in some embodiments, the constraining member 340 can be a clip, a band, a fastener, a hook, a clamp, an adhesive, and/or any other suitable device or combination thereof.

As shown in FIG. 3B, the actuator 321 can be actuated to move to its second configuration. In some embodiments, the actuation of the actuator 321 corresponds to the discharging of the electrochemical cell defined thereby. The discharging of the electrochemical cell can be such that the medial portion 324 bends, thereby producing an angular deflection of the first end portion 322 and the second end portion 323 relative to the medial portion 324. Similarly stated, the constrained medial portion 324 can bend such that the unconstrained first end portion 322 and the unconstrained second end portion 323 deflect relative to the constrained medial portion 324. Thus, the sum of the angular deflection of the first end portion 322 and the angular deflection of the second end portion 323 can define the total angular deflection Θ₁ of the actuator 321.

In some embodiments, the actuator 321 can be arranged relative to the constraining member 340 such that the first end portion 322 and the second end portion 323 are equidistant from the constraining member 340 and/or the medial portion 324. In this manner, the angular deflection of the first end portion 322 can be the same as the angular deflection of the second end portion 323. Furthermore, the medial portion 340 can exert a first force F₁ in a first direction on an adjacent structure (e.g., a portion of a delivery device as described in further detail herein) and the first end portion 322 and the second end portion 323 can each exert a second force F₂ in a second direction, opposite the first direction, on an adjacent structure. With the first end portion 322 and the second end portion 323 equidistant from the constrained medial portion 324, the second force F₂ exerted by both the first end portion 322 and the second end portion 323 collectively equal the first force F₁ exerted by the constrained medial portion 324. Moreover, the deflection of both the first end portion 322 and the second end portion 323 results in a change in height Δh₃ of the actuator 321, as shown in FIG. 3B. Thus, by constraining the medial portion 324 (e.g., via the constraining member 340), the actuator 321 can exert an evenly distributed load on a structure such as, for example, a surface of a fluid reservoir, a further described herein with respect to specific embodiments.

While the actuator 321 is shown in FIGS. 3A and 3B as having the first end portion 322 and the second end portion 323 equidistant from the constraining member 340, in other embodiments the first end portion 322 and the second end portion 323 can be different distances from the constraining member 340. In this manner, the actuator 321 can be configured to exert a non-uniform load on an adjacent structure that may be suitable, for example, in use with specific fluid reservoirs.

While the constraining member 340 is shown in FIGS. 3A and 3B as constraining one actuator 321, in other embodiments a constraining member can constrain more than one actuator. For example, FIGS. 4A and 4B illustrate a first actuator 421 and a second actuator 421′ collectively constrained by a constraining member 440 in a first configuration and a second configuration, respectively, according to an embodiment. The first actuator 421 includes a first end portion 422, a second end portion 423, and a medial portion 424. Similarly, the second actuator 421′ includes a first end portion 422′, a second end portion 423′, and a medial portion 424′. In some embodiments, the first actuator 421 can be the same as the second actuator 421′ and both the first actuator 421 and the second actuator 421′ can be similar in form and function to the actuator 321 described above with reference to FIGS. 3A and 3B. Therefore, the first actuator 421 and the second actuator 421′ are not described in further detail herein.

As shown in FIG. 4A, the first actuator 421 and the second actuator 421′ can be substantially planar when in the first configuration (e.g., a charged configuration) and can collectively define a height h₄. In addition, the first actuator 421 and the second actuator 421′ can be collectively arranged such that the constraining member 440 constrains the medial portion 424 of the first actuator 421 and the medial portion 424′ of the second actuator 421′. Thus, when the first actuator 421 and/or the second actuator 421′ are moved to the second configuration (FIG. 4B), the position of the medial portion 424 of the first actuator 421 relative to the position of the medial portion 424′ of the second actuator 421 is retained, as further described herein.

As shown in FIG. 4B, the medial portion 424 of the first actuator 421 and the medial portion 424′ of the second actuator 421′ can bend to move the first actuator 421 and the second actuator 421′, respectively, from the first configuration to the second configuration. In this manner, the first end portion 422 and the second end portion 423 of the first actuator 421 can deflect relative to the medial portion 424. Similarly stated, the constrained medial portion 424 (e.g., by the constraining member 440) can bend to produce an angular deflection of the unconstrained first end 422 and the unconstrained second end 423 relative to the medial portion 424. Thus, the sum of the angular deflection of the first end portion 422 and the second end portion 423 can define the total angular deflection Θ₂ of the actuator 421. Similarly, the constrained medial portion 424′ of the second actuator 421′ can bend to produce an angular deflection of the unconstrained first end portion 422′ and the unconstrained second end portion 423′ such that the sum of the angular deflections define a total angular deflection Θ₃ of the second actuator 421′. Moreover, with the first actuator 421 and the second actuator 421′ being similar, the angular deflections Θ₂ and Θ₃ can be substantially similar.

In some embodiments, the angular deflection Θ₂ of the first actuator 421 and the angular deflection Θ₃ of the second actuator 421′ produce a change in height Δh₄ of both first actuator 421 and the second actuator 421′. For example, the first end portion 422 and the second end portion 423 of the first actuator 421 can be disposed adjacent to a structure (e.g., of a delivery device, as described in further detail herein) such that as the first end portion 422 and the second end portion 423 deflect, the medial portion 424 is moved away from the adjacent structure. Similarly, the first end portion 422′ and the second end portion 423′ of the second actuator 421′ can be disposed adjacent to a second structure such that as the first end portion 422′ and the second end portion 423′ deflect, the medial portion 424′ is moved away from the adjacent structure. Thus, when disposed adjacent to a movable structure, the change in height Δh₄ of the first actuator 421 and the second actuator 421′ can move at least a portion of the movable structure. For example, in some embodiments, the first actuator 421 or the second actuator 421′ can be disposed adjacent to a fluid reservoir pouch and the change in height Δh₄ can move a portion of the pouch to dispense a fluid disposed therein, as described in further detail herein.

While the first actuator 421 and the second actuator 421′ are described as being substantially similar, in other embodiments the first actuator 421 and the second actuator 421′ can be different. For example, in some embodiments, the first actuator 421 can define a first electrochemical composition and the second actuator 421′ can define a second electrochemical composition, different from the first. Thus, the first actuator 421 can be configured to discharge (e.g., move to the second configuration) with a first set of characteristics (e.g., angular deflection, change of height, exerted force, rate of discharge, or a combination thereof) and the second actuator 421′ can be configured to discharge with a second set of characteristics, different from the first. For example, in some embodiments, the first actuator 421 can have a first angular deflection that is less than the angular deflection of the second actuator 421′. In other embodiments the first actuator 421 can be configured to discharge at a faster rate than the second actuator 421′. In this manner, the actuators 421 and 421′ can be configured to discharge with any suitable collective characteristics.

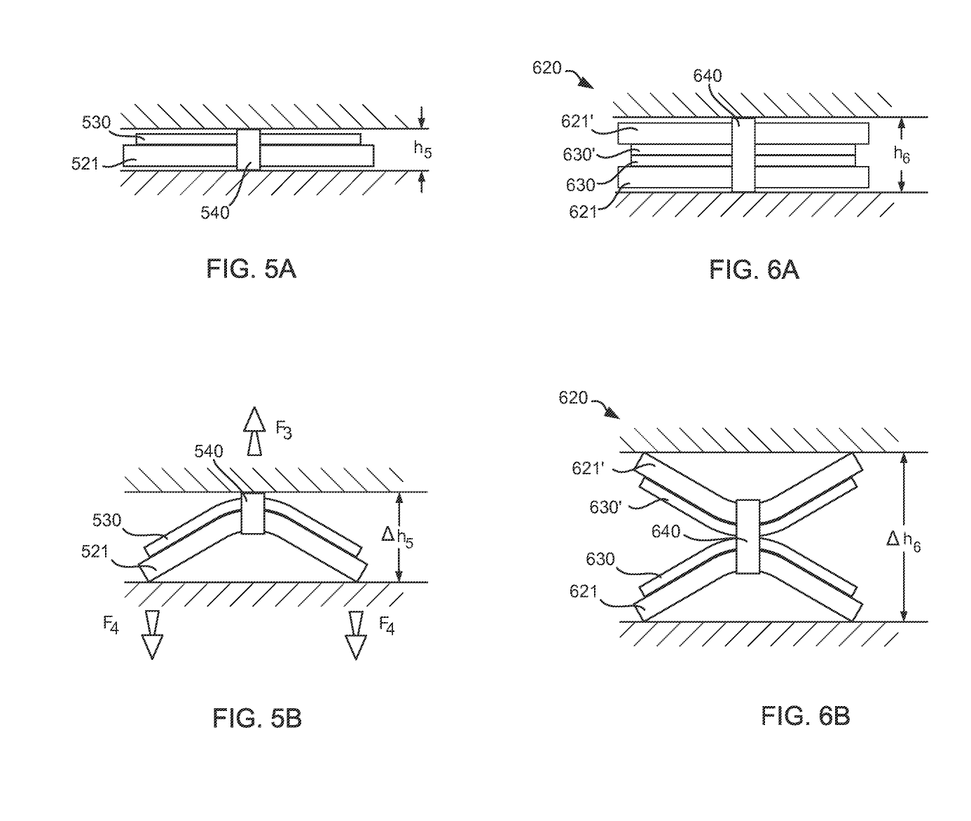

While the first actuator 421 and the second actuator 421′ are described above as being electrochemical actuators, in other embodiments, an electrochemical actuator can be used in conjunction with a mechanical actuator. For example, FIGS. 5A and 5B illustrate a first actuator 521 and a second actuator 530 in a first configuration and a second configuration, respectively, according to an embodiment. As shown in FIG. 5A, the first actuator 521 and the second actuator 530 can be collectively constrained by a constraining member 540. In some embodiments, the first actuator 521 can be an electrochemical actuator substantially similar to the electrochemical actuator 321 described above with reference to FIGS. 3A and 3B. Thus, the first actuator 521 is not described in further detail herein. The second actuator 530 can be a mechanical actuator such as a spring (e.g., a leaf spring, a compression spring, a Bellville spring, or the like).

As shown in FIG. 5A, the clamping mechanism 540 can be configured to retain at least a portion of the first actuator 521 relative to the second actuator 530, such that the first actuator 521 and the second actuator 530 are substantially planar when in the first configuration. More specifically, while in the first configuration, the first actuator 521 can be configured to maintain the second actuator 530 in its first configuration. For example, in some embodiments, the first actuator 521 can exert a reaction force on at least a portion of the second actuator 530 that is sufficiently large to hold the spring is in a high potential energy configuration). Thus, the first actuator 521 and the second actuator 530 can be in the first configuration until the first actuator 521 is at least partially discharged (e.g., begins to move towards the second configuration). Moreover, the first actuator 521 and the second actuator 530 collectively define a height h₅ associated with the first configuration. Once the first actuator 521 begins to move from the first configuration to the second configuration, the second actuator 530 can begin to convert the stored potential energy to kinetic energy, thereby exerting a force on the first actuator 521.

As shown in FIG. 5B, the first actuator 521 and the second actuator 530 can collectively move from the first configuration to the second configuration such that a medial portion of both the first actuator 521 and the second actuator 530 bends. In this manner, at least a portion (e.g., each end portion) of the first actuator 521 can be configured to move substantially perpendicularly to its constrained medial portion. Similarly, at least a portion of the second actuator 530 can be configured to move substantially perpendicularly to its constrained medial portion. In this manner, the medial portion of the first actuator 521 and the medial portion of the second actuator 530 can collectively exert a first force F₃ in a first direction on an adjacent structure and the end portions of the first actuator 521 and/or of the second actuator 530 can each exert a second force F₄ in a second direction, opposite the first, on an adjacent structure. Expanding further, when the first actuator 521 is moved from the first configuration, the reaction force maintaining the second actuator 530 in its first configuration is removed. Thus, the second actuator 530 is allowed to move to its second configuration. In this manner, the second actuator 530 exerts a force on a portion of the first actuator 521 to increase the collective force exerted on the adjacent structures.

The deflection of the end portions of the first actuator 521 and the second actuator 530 result in a change in height Δh₅ of the first actuator 521 and the second actuator 530, as shown in FIG. 5B. Thus, the first actuator 521 and the second actuator 530 can move to the second configuration to move an adjacent structure such as, for example, a portion of a fluid reservoir. Moreover, the addition of the second actuator 530 (e.g., a mechanical actuator) can increase the force exerted on the adjacent structure without substantially increasing the overall change in height Δh₅. Therefore, such embodiments can be suitable in, for example, delivery devices with a low profile.

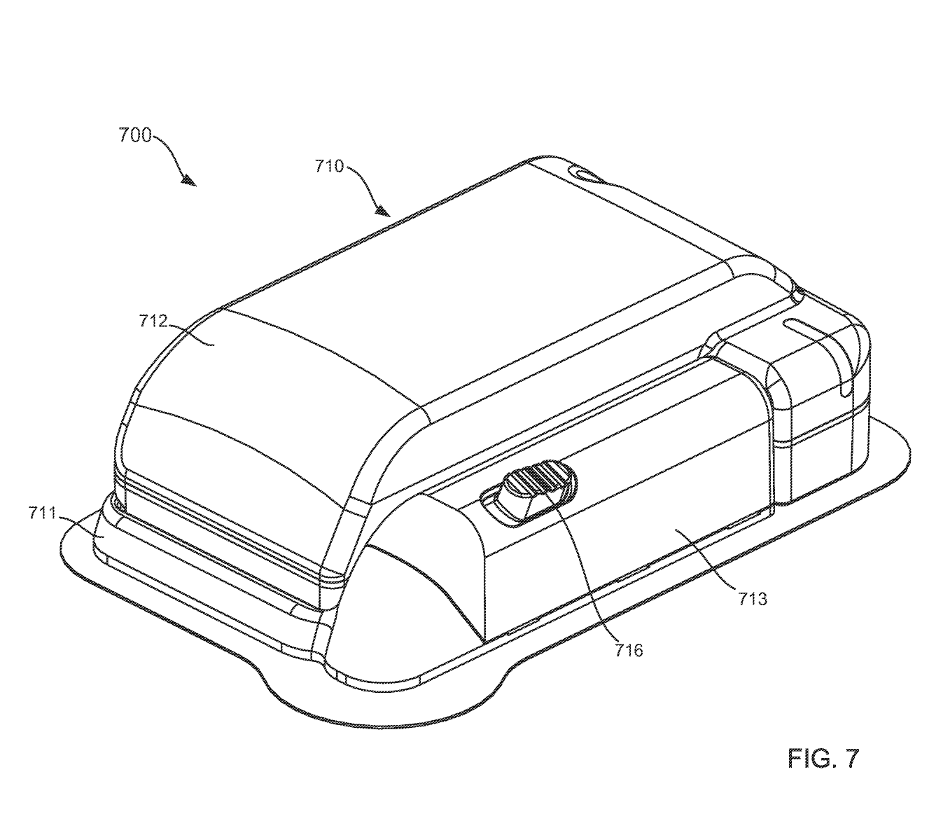

While the constraining member 540 is shown in FIGS. 5A and 5B as constraining two actuators (e.g., the first actuator 521 and the second actuator 530), in other embodiments, a constraining member can constrain more than two actuators. For example, FIGS. 6A and 6B illustrate an actuator assembly 620 in a first configuration and a second configuration, respectively, according to an embodiment. The actuator assembly 620 includes a first electrochemical actuator 621, a second electrochemical actuator 621′, a first mechanical actuator 630, a second mechanical actuator 630′, and a constraining member 640. The electrochemical actuators 621 and 621′ and the mechanical actuators 630 and 630′ can be similar in form and function as the electrochemical actuator 521 and the mechanical actuator 530 described above with reference to FIGS. 5A and 5B. Therefore, the electrochemical actuators 621 and 621′ and the mechanical actuators 630 and 630′ are not described in further detail herein.

As shown in FIG. 6A, the clamping mechanism 640 can be configured to retain a portion of the actuators 621, 621′, 630 and 630′ such that the actuators 621, 621′, 630 and 630′ are substantially planar when in the first configuration. More specifically, while in the first configuration, the first electrochemical actuator 621 and the second electrochemical actuator 621′ can be configured to maintain the first mechanical actuator 630 and the second mechanical actuator 630′ in the first configuration, as described above. Thus, the electrochemical actuators 621 and 621′ and the mechanical actuators 630 and 630′ can be in the first configuration until the electrochemical actuators 621 and 621′ are at least partially discharged (e.g., begin to move towards the second configuration). Moreover, the electrochemical actuators 621 and 621′ and the mechanical actuators 630 and 630′ collectively define a height h₆ associated with the first configuration.

As shown in FIG. 6B, the electrochemical actuators 621 and 621′ and the mechanical actuators 630 and 630′ can collectively move from the first configuration to the second configuration such that at least a medial portion of the actuators 621, 621′, 630, and 630′ can bend. In this manner, at least a portion (e.g., each end portion) of the actuators 621, 621′, 630, and 630′ can be configured to move substantially perpendicularly to the constrained medial portion. The deflection of the actuators 621, 621′, 630, and 630′ is configured to produce a change in height Δh₆ of actuator assembly 620. For example, the first electrochemical actuator 621 can be disposed adjacent to a structure (e.g., of a delivery device, as described in further detail herein) such that as the end portions deflect, the medial portion is moved away from the adjacent structure. Similarly, the second electrochemical actuator 621′ can be disposed adjacent to a second structure such that as the end portions deflect, the medial portion is moved away from the adjacent structure (e.g., toward the first electrochemical actuator 621). Thus, when disposed adjacent to a movable structure, the change in height Δh₆ of the actuator assembly 620 can move at least a portion of the movable structure. For example, in some embodiments, the first actuator 621 or the second actuator 621′ can be disposed adjacent to a fluid reservoir pouch such that the change in height Δh₅ can move a portion of the pouch to dispense a fluid disposed therein, as described in further detail herein. In addition, the mechanical actuators 630 and 630′ can be configured to increase the force exerted on the adjacent structures, as described above.

FIGS. 7-12 illustrate an embodiment of a delivery device that can include at least one electrochemical actuator as described herein. A delivery device 700 includes a housing 710 configured to house an insertion assembly, an actuator assembly 720, a fluid reservoir 780, and an electronic assembly 718. The housing 710 can be formed from a material that is relatively lightweight and flexible, yet sturdy. The housing 710 also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

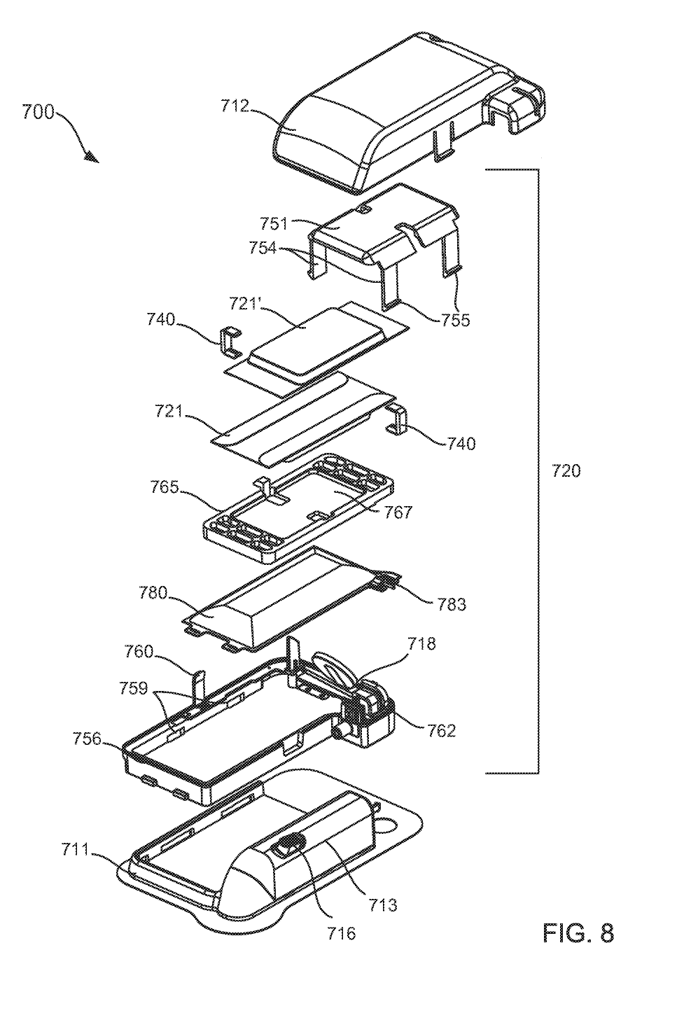

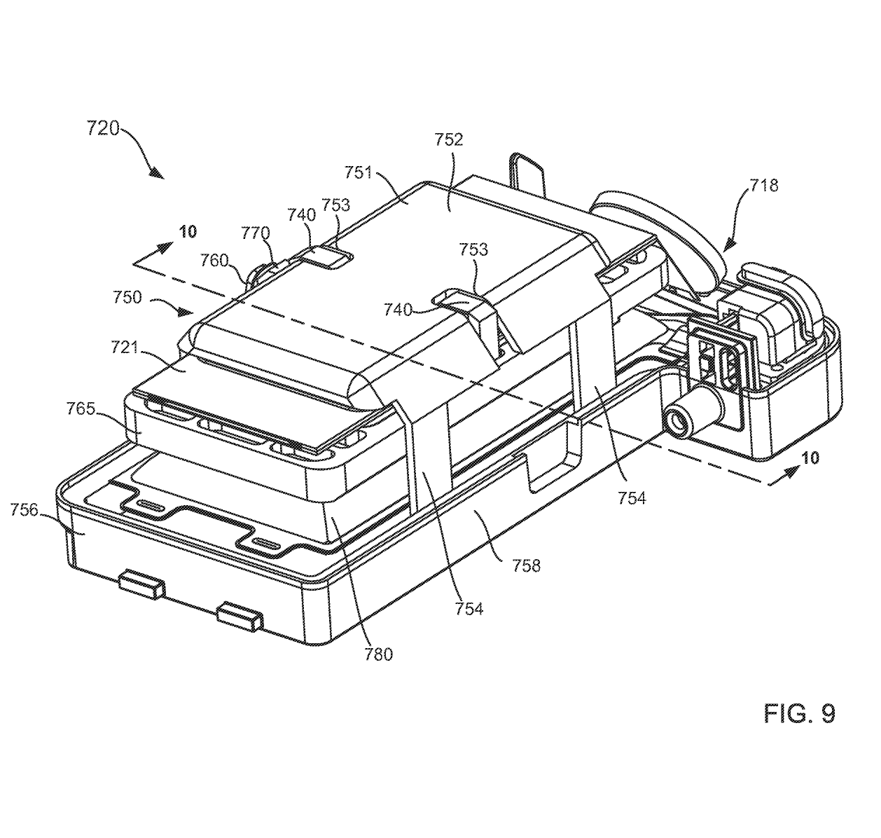

In some embodiments, the housing 710 can include a single component or multiple components. For example, as shown in FIG. 8, the housing 710 can include a first portion 711, a second portion 712, and a third portion 713. The first portion 711 can be, for example, a base portion suitable for attaching to the skin of a patient. For example, the first portion 711 can be relatively flexible. In some embodiments, an adhesive can be deposited on an underside of the first portion 711, which can be relatively flat or shaped to conform to the shape of a particular body part or area.

The second portion 712 can be any suitable size or shape. For example, in some embodiments, the size and shape of the second portion 712 can be associated with the first portion 711. In some embodiments, the first 711 portion and the second portion 712 can be designed to lock together, such as via a locking mechanism. In some cases, the first portion 711 and the second portion 712 can releasably lock together, such as via a releasable locking mechanism (e.g., one or more latches, one or more tabs, or the like), so that the second portion 712 can be removably coupled to the first portion 711. For example, to assemble such a housing 711, the second portion 712 can be movable with reference to the first portion 711 between an unassembled position and an assembled position. In the assembled position, the first portion 711 and the second portion 712 can define an inner volume configured to house the actuator assembly 720, the fluid reservoir 780, and at least a portion of the electronics assembly 718.

The third portion 713 of the housing 710 is configured to be removably coupled to the first portion 711. For example, in some embodiments, the third portion 713 can be removably coupled to the first portion 711 in a similar manner as the second portion 712. Thus, the third portion 713 can be movable with reference to the first portion 711 between an unassembled position and an assembled position. In the assembled position, the third portion 713 and the first portion 711 can define an inner volume configured to house at least a portion of the insertion assembly. In this manner, the housing 710 can have an outer shape suited for concealing the device under clothing. Various example embodiments of a housing 710 are described in the '771 patent.

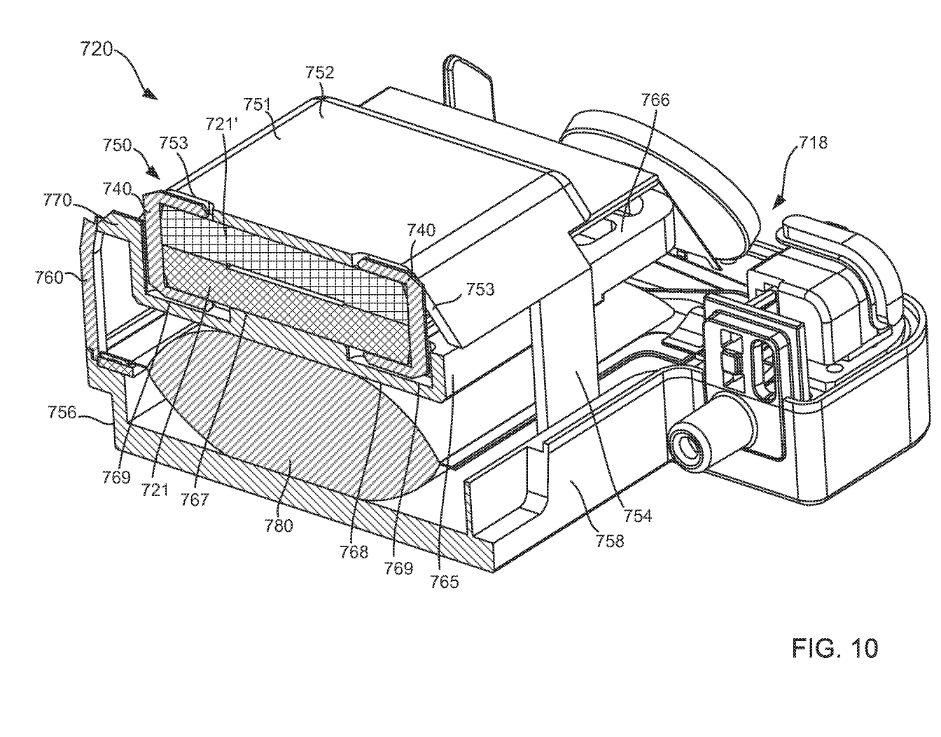

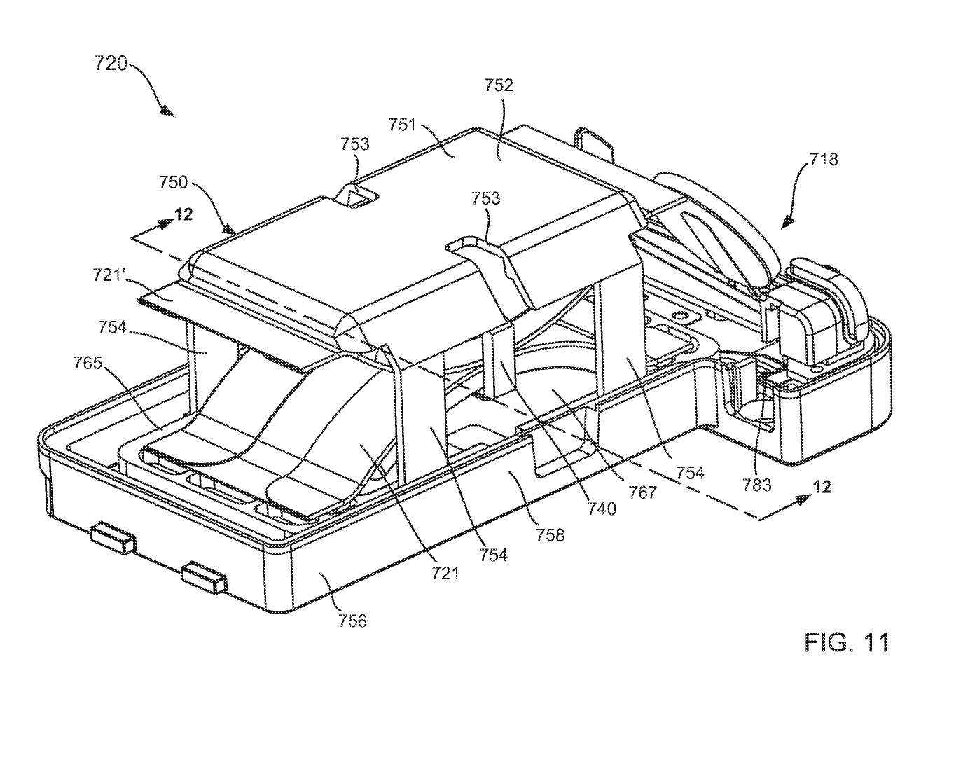

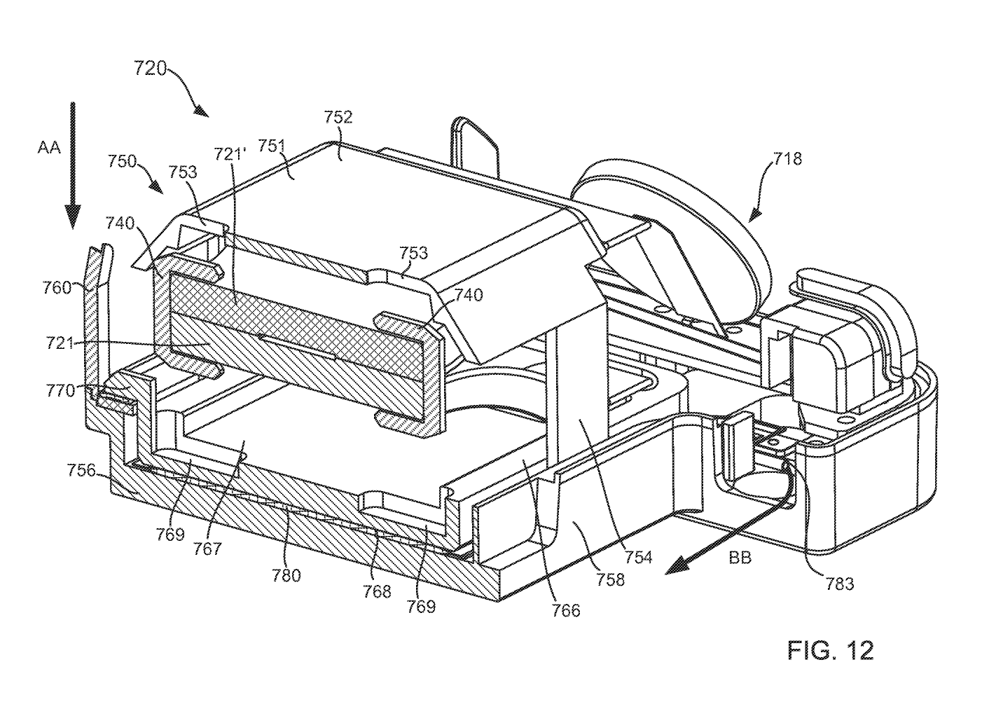

As described above, the actuator assembly 720 is disposed within the inner volume of the housing 710 (e.g., defined by the first portion 711 and the second portion 712) and is configured to move between a first configuration (FIGS. 9 and 10) and a second configuration (FIGS. 11 and 12). The actuator assembly 720 includes a first actuator 721, a second actuator 721′, a clamping mechanism 740, a support structure 750, and a transfer structure 765 (see e.g., FIGS. 8 and 9). The first actuator 721 and the second actuator 721′ can be any suitable actuators described herein. For example, in some embodiments, the first actuator 721 and the second actuator 721′ can each be an electrochemical actuator and can be substantially similar to the electrochemical actuator 221 described above with reference to FIGS. 2A and 2B. In this manner, the first actuator 721 and the second actuator 721′ can be configured to move between a first configuration and a second configuration in response to a change in an electrical state. Expanding further, while in the first configuration, at least a portion of the first actuator 721 and at least a portion of the second actuator 721′ are substantially planar and, when moved to the second configuration, at least a portion of the first actuator 721 and at least a portion of the second actuator 721′ can deflect to produce a change in overall height of the first actuator 721 and the second actuator 721′, respectively.

The first actuator 721 and the second actuator 721′ can be arranged such that the first actuator 721 is disposed adjacent to the second actuator 721′ but facing opposite directions. Similarly stated, the first actuator 721 and the second actuator 721′ can be arranged in a back-to-back configuration. In this manner, the first actuator 721 and the second actuator 721′ can be configured to deflect in opposite directions, as described in further detail herein.

The constraining member 740 is configured to engage at a portion of the first actuator 721 and a portion of the second actuator 721′ (see e.g., FIG. 10). For example, as shown, the constraining member 740 can be a C-shaped clamp configured to receive the portion of the first actuator 721 and the portion of the second actuator 721′. In some embodiments, the constraining member 740 can be configured such that a height defined between a set of arms (e.g., forming the C-shape) is smaller than a collective height of the first actuator 721 and the second actuator 721′. Thus, the constraining member 740 can form a friction fit with the portion of the first actuator 721 and the portion of the second actuator 721′.

The support structure 750 includes a first member 751 and a second member 756 that can be coupled together to retain the first actuator 721, the second actuator 721′, and the transfer structure 765 therebetween. More specifically, the first member 751 includes a planar portion 752 and a set of extensions 754 that extend from the planar portion 752. The planar portion 752 is configured to be in contact with at least a portion of the second actuator 721′. The planar portion 752 is further configured to define a set of notches 753. The notches 753 can receive a portion of the constraining member 740, when the actuator assembly 720 is in its first configuration. Expanding further, by disposing the portion of the constraining member 740 within the notches 753, the distance between the planar portion 752 of the first member 751 and the transfer structure 765 can be minimized, thereby reducing the overall size of the delivery device 700.

The extensions 754 can include a set of tabs 755 that can be disposed within a set of slots 759 defined by one or more walls 758 of the second member 756 (see e.g., FIGS. 8 and 9). In this manner, the first member 751 can be moved relative to the second member 756 such that the tabs 755 are disposed within the slots 759, thereby coupling the first member 751 to the second member 756. The arrangement of the tabs 755 within the slots 759 can be such that the first member 751 is at least temporarily fixedly coupled to the second member 756. Similarly stated, the first member 751 can be coupled to the second member 756 such that the first member 751 substantially does not move relative to the second member 756.

As shown in FIG. 9, the fluid reservoir 780 is configured to be disposed on and/or supported by the second member 756 of the support structure 751. Moreover, the walls 758 of the second member 756 can be configured to substantially limit a lateral movement of the fluid reservoir 780. The second member 756 is further configured to include a status window 760. The status window 760 can be, for example, a substantially transparent portion of the second member 756 through which a user can visualize the status of the delivery device 700 (e.g., the level of the fluid reservoir or the like).

The fluid reservoir 780 can be provided to a user predisposed within the inner volume of the housing 710 or can be provided as a separate component that the user can insert into the housing 710. For example, the fluid reservoir 780 can be inserted through an opening (not shown) in the housing 710. The fluid reservoir 780 can be any suitable reservoir. For example, in some embodiments, the fluid reservoir 780 can be a bag, a flexible container, a pouch, etc. that defines an interior volume that can contain a fluid to be injected into a patient. The fluid reservoir 780 can include a port 783 (FIG. 8) configured to be punctured by the insertion mechanism (not shown) to create a fluid channel between the fluid reservoir 780 and a fluid communicator (not shown) configured to penetrate the patient's skin. In some embodiments, the fluid reservoir 480 can be sized for example, with a length of about 2 cm, a width of about 2 cm, and a height of about 0.25 cm, to contain, for example, a total volume of 1 ml of fluid.

The transfer structure 765 of the actuator assembly 720 is movably disposed between the first actuator 721 and the fluid reservoir 780, as further described herein. As shown in FIG. 10, the transfer structure 765 includes a first surface 766 configured to engage the first actuator 721 and a second surface 768 configured to engage the fluid reservoir 780. More specifically, the first surface 766 of the transfer structure 765 defines a recess 767 and a pair of notches 769. The recess 767 is configured to receive a portion of the first actuator 721 and the notches 769 are configured to receive the constraining member 740, as shown in FIG. 10. In this manner, the distance between the transfer structure 765 and the planar portion 752 of the first member 751 can be further minimized (as described above). Therefore, in some embodiments, the overall height of the delivery device can be minimized. In other embodiments, the additional space provided by disposing the first actuator 721 in the recess 767 and the constraining member 740 in the notches 769 can allow for the inclusion of a fluid reservoir of larger volume than would otherwise be suitable. The transfer structure 765 can further include an indicator member 770. The indicator member 770 can be disposed adjacent to the status window 760 of the second member 756 and can provide a visual status indication to the user.

To use the delivery device 700, the delivery device 700 is placed at a desired injection site on a patient's body and adhesively attached thereto. With the fluid reservoir 780 disposed within the housing 710 (e.g., inserted into the housing 710 by the patient or predisposed), the patient can activate the insertion mechanism (not shown) to insert a fluid communicator (not shown) at the injection site. To activate the insertion mechanism to insert the fluid communicator (not shown) into a patient's body, an activation mechanism 716 (e.g., a button included in or coupled to the insertion mechanism) can be moved from an off position to an on position such that the fluid communicator included in the insertion mechanism penetrates the skin of the patient at the treatment site. Furthermore, the activation of the insertion mechanism can be such that a portion (not shown) of the insertion mechanism punctures the port 783 of the fluid reservoir 780 to define a fluid channel between the fluid reservoir 780 and the fluid communicator (not shown).

In some embodiments, the actuator assembly 720 can be activated after the insertion mechanism has been activated and the fluid communicator has been inserted into the patient's body. Alternatively, in some embodiments, the actuator assembly 720 can be activated simultaneously with activation of the insertion mechanism. For example, when the insertion mechanism is activated a trigger mechanism (not shown) can be activated that communicates with the actuator assembly 720. For example, such a trigger mechanism can complete (e.g., close) an electric circuit included in the electronic system 718 to cause the first actuator 721 and/or second actuator 721′ to start discharging.

The discharging of the first actuator 721 and/or the second actuator 721′ can be such that the actuator assembly 720 is moved from the first configuration (FIGS. 9 and 10) to the second configuration (FIGS. 11 and 12). As described above, the discharging of the first actuator 721 and/or the second actuator 721′ corresponds to a deflection of at least a portion of the first actuator 721 and/or at least a portion of the second actuator 721′, respectively. For example, as shown in FIGS. 11 and 12, at least a portion of the first actuator 721 and at least a portion of the second actuator 721′ move substantially perpendicularly relative to the portion of the first actuator 721 and the second actuator 721′, respectively, constrained by the constraining member 740 (described above).

The deflection of the second actuator 721′ is such that the constrained portion (e.g., the portion constrained by the constraining member 740 referred to herein as a medial portion) is moved in a first direction towards the first actuator 721. Expanding further, with the first member 751 of the support structure 750 coupled to the second portion 756 (as described above) and with at least a portion of the second actuator 721′ in contact with the planar portion 752 of the first member 751, the discharging of the second actuator 721′ is such that the medial portion of the second actuator 721′ deflects in the first direction. Similarly stated, during discharge of the second actuator 721′ the end portions exert a force on the first member 751 of the support structure 750, which in turn, exerts an equal but opposite reaction force on the end portions. Thus, the second actuator 721′ does work (e.g., exerts a force) to deflect the medial portion in the first direction.

In a similar manner, the first actuator 721 does work to deflect the medial portion in the second direction, opposite the first direction. However, the deflection of the first actuator 721 is configured to displace the transfer structure 765 relative to the support structure 750. Expanding further, with the constraining member 740 constraining the medial portion of the first actuator 721 relative to the medial portion of the second actuator 721′ the force exerted by the second actuator 721′ moves the medial portion of the second actuator 721′ and the medial portion of the first actuator 721 in the first direction, as indicated by the arrow AA in FIG. 12. Thus, the constraining member 740 (and therefore, the medial portions of the actuators 721 and 721′) is moved away from the first member 751 of the support structure 750. Furthermore, with the transfer structure 765 being movable relative to the support structure 750, the force exerted by the first actuator 721 and at least a portion of the force exerted by the second actuator 721′ moves the transfer structure 765 in the direction of the arrow AA. Thus the transfer structure 765 is moved toward the second member 756 of the support structure 750.

As shown in FIG. 12, the movement of the transfer structure 765 is such that the at least a portion of the force exerted by the first actuator 721 and the second actuator 721′ is transferred to the fluid reservoir. As described above, the fluid reservoir 780 can be a flexible reservoir such as a pouch or bag. Therefore, with the fluid reservoir 780 in fluid communication with the fluid communicator, the force exerted on the fluid reservoir 780 by the transfer structure 765 displaces a portion of the fluid reservoir 780, increasing the pressure therein. Thus, the fluid disposed within the fluid reservoir 780 is urged to exit the port 783, as indicated by the arrow BB in FIG. 12. Moreover, with the insertion mechanism in fluid communication with the port 783 (as described above), the fluid can flow within a fluid channel defined between the fluid reservoir 780 and the fluid communicator (not shown) and can exit the fluid communicator to be delivered to the target site.

In some embodiments, the inclusion of the support structure 750 can be configured contain the forces exerted by the actuators 721 and 721′. For example, in some embodiments, the support structure 750 can contain the forces exerted by the actuators 721 and 721′ such that the forces are not transferred to the housing 710. Therefore, when the delivery device 700 is worn on the skin, the forces are not transferred to the patient (e.g., the patient does not feel the forces exerted by the actuators 721 and 721′).

While not shown in FIGS. 7-12, in some embodiments, a delivery device can include one or more mechanical actuators that can be used in conjunction with the electrochemical actuators. For example, in some embodiments, a delivery device can include a first mechanical actuator and a second mechanical actuator. In such embodiments, the mechanical actuators can be, for example, leaf springs or the like. In this manner, the actuators (i.e., the two electrochemical actuators and the two mechanical actuators) can be arranged in a configuration that is substantially similar to that shown in FIGS. 6A and 6B. In other embodiments, a delivery device can include any other suitable spring such as, for example, a compression spring, a torsion spring, a Bellville spring In this manner, the mechanical actuators can be configured to increase the amount of force exerted on the fluid reservoir while maintaining a similar stroke length (e.g., the same change in height or the same amount of deflection). Thus, the delivery device can be used with fluids (e.g., medicaments) having a relatively high viscosity.

While the second surface 768 of the transfer device 765 is shown as being substantially flat, in other embodiments, the second surface 768 can be any suitable configuration. For example, in some embodiments, the second surface 768 can be angled such that the transfer structure 760 is substantially wedged shape. In other embodiments, the second surface 768 can be curvilinear. In this manner, the transfer member 765 can be configured to selectively engage the fluid reservoir 780 to exert at least a portion of the force from the actuators 721 and 721′ on the fluid reservoir 780. While the transfer device 765 is described as moving in a single direction, in other embodiments, the transfer structure can be moved in more than one direction. For example, in some embodiments, the transfer structure 765 can be moved along a curved path. In this manner, a first portion of the transfer structure 765 can engage the fluid reservoir 780 prior to a second portion engaging the fluid reservoir 780. For example, in some embodiments, it may be desirable to engage an end of the fluid reservoir 780 that is opposite the end including the port 783 to ensure complete delivery of the fluid contained therein.

While described as discharging at about the same time, in some embodiments, the first actuator 721 can be configured to discharge prior to the second actuator 721′ (or vice versa). In such embodiments, it may be desired to partially displace the portion of the fluid reservoir 780 to initiate, for example, a mixing of a medicament disposed therein prior to the port 783 being punctured. In such embodiments, the discharge of the other actuator can additionally displace the portion of the fluid reservoir 780 to urge the fluid to flow through the port 783.

Although the delivery devices described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may deliver drugs or other fluids into living organisms other than human beings, such as animals and plant life. Also, the systems and methods may deliver any fluids into any target, living or inanimate.

Any delivery device described herein can be used to deliver a variety of drugs according to one or more release profiles. For example, in some embodiments, a delivery device can be operated with a controller and/or other circuitry, operative to regulate drug or fluid flow from the delivery device. Such a controller may permit implementing one or more release profiles using the pump device, including release profiles that require uniform flow, non-uniform flow, continuous flow, discontinuous flow, programmed flow, scheduled flow, modulated flow, user-initiated flow, feedback responsive flow, among others. Thus, the delivery device can be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

For example, any of the delivery devices described herein can be useful in a wide variety of therapies such as, but not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine, and bupivicaine; dopamine antagonists such as apomorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline; any peptide, protein, biologic, or oligonucleotide, among others, that is normally delivered by subcutaneous, intramuscular, or intravenous injection or other parenteral routes. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, a delivery device can be used to administer ketamine for the treatment of refractory depression or other mood disorders. In some embodiments, ketamine can include either the racemate, single enantiomer (R/S), or the metabolite (wherein S-norketamine may be active). In some embodiments, the delivery devices described herein can be used for administration of Interferon A for the treatment of hepatitis C. In one embodiment, a several hour infusion patch is worn during the day or overnight three times per week, or a continuous delivery system is worn 24 hours per day. Such a delivery device may advantageously replace bolus injection with a slow infusion, reducing side effects and allowing the patient to tolerate higher doses. In other Interferon A therapies, the delivery device can also be used in the treatment of malignant melanoma, renal cell carcinoma, hairy cell leukemia, chronic hepatitis B, condylomata acuminata, follicular (non-Hodgkin's lymphoma, and AIDS-related Kaposi's sarcoma.

In some embodiments, any delivery device described herein can be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. In this manner, any of the delivery devices described herein can be used to provide continuous delivery of apomorphine that may dramatically reduce side effects associated with both apomorphine and dopamine fluctuation. In some embodiments, a delivery device as described herein can provide continuous delivery of apomorphine or other dopamine agonist with, optionally, an adjustable baseline and/or a bolus button for treating an “off” state in the patient. Such a method of treatment can provide improved dopaminergic levels in the body, such as fewer dyskinetic events, fewer “off” states, less total time in “off” states, less cycling between “on” and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

While various embodiments are described herein, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the embodiments. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. For example, although some embodiments were not described as including an insertion mechanism, an activation mechanism, electrical circuitry, etc., it should be understood that those embodiments of a delivery device can include any of the features, components and/or functions described herein for other embodiments. In addition, the specific configurations of the various components can also be varied. For example, the size and specific shape of the various components can be different than the embodiments shown, while still providing the functions as described herein.

1. An apparatus, comprising:
a reservoir configured to contain a fluid;
a fluid communicator configured to be placed in fluid communication with the reservoir; and
an actuator having a first end, a second end, and a medial portion between the first end and the second end, the actuator being configured so that when actuated, the actuator bends in the medial portion and produces a displacement of the second end in a first direction relative to the first end,
the actuator being disposed with the first end constrained, the second end unconstrained, and oriented so that when the actuator is actuated, the second end is displaced toward the fluid reservoir and exerts a force on the reservoir such that fluid within the reservoir is communicated through the fluid communicator.

2. The apparatus of claim 1, further comprising:
a transfer structure disposed between the actuator and the reservoir, the transfer structure configured to contact the reservoir upon actuation of the actuator.

3. The apparatus of claim 1, wherein the actuator is an electrochemical actuator.

4. The apparatus of claim 1, further comprising:
a housing configured to be removably coupled to a patient, the housing defining an interior region, the actuator and reservoir disposed within the housing.

5. The apparatus of claim 1, further comprising:
a housing configured to be removably coupled to a patient; and
an insertion mechanism coupled to the housing, the insertion mechanism configured to insert the fluid communicator into the patient.

6. The apparatus of claim 1, further comprising:
a transfer structure disposed between the actuator and the reservoir, the transfer structure having a top surface configured to contact the reservoir upon actuation of the actuator, the top surface of the transfer structure being non-parallel to a top surface of the actuator.

7. The apparatus of claim 1, wherein the reservoir is wedge shaped prior to actuation of the actuator.

8. An apparatus, comprising:
a housing removably couplable to a user;
a reservoir configured to contain a fluid and disposed within the housing;
an actuator having a constrained first end portion and an unconstrained second end portion; and
a transfer structure disposed between the actuator and the reservoir, the transfer structure having a first end portion pivotally coupled to the housing and an unconstrained second end portion, the transfer structure having a surface configured to contact the reservoir upon actuation of the actuator,
the actuator being configured such that when actuated, a force is exerted by the second end portion of the actuator onto the transfer structure and the unconstrained second end portion of the transfer structure pivots about a pivot location and exerts a force on the reservoir such that fluid within the reservoir is communicated out of the reservoir.

9. The apparatus of claim 8, wherein the actuator is an electrochemical actuator.

10. The apparatus of claim 8, further comprising:
a fluid communicator configured to be placed in fluid communication with the reservoir such that when the actuator is actuated, fluid in the reservoir is communicated into the user via the fluid communicator.

11. The apparatus of claim 8, wherein the actuator is a first actuator, the force exerted by the actuator is a first force, the apparatus further comprising:
a second actuator having a constrained first end portion and an unconstrained second end portion, the second actuator configured to exert a second force, different than the first force, on the reservoir.

12. The apparatus of claim 11, wherein the transfer structure is a first transfer structure, the apparatus further comprising:
a second transfer structure disposed between the second actuator and the reservoir, the second actuator configured to exert the second force on the second transfer structure in an opposite direction as the first force.

13. The apparatus of claim 11, wherein the transfer structure is a first transfer structure, the apparatus further comprising:
a second transfer structure disposed between the second actuator and the reservoir, the second transfer structure having a first end portion pivotally coupled to the housing and an unconstrained second end portion, the second transfer structure having a surface configured to contact the reservoir upon actuation of the actuator,
the second actuator being configured such that when actuated, the second force is exerted by the second end portion of the actuator onto the transfer structure and the unconstrained second end portion of the transfer structure pivots about a pivot location and exerts a force on the reservoir such that fluid within the reservoir is communicated out of the reservoir.

14. The apparatus of claim 8, wherein the force exerted by the actuator is a first force, the apparatus further comprising:
a spring coupled to the transfer structure, the spring configured to exert a second force onto the transfer structure such that the unconstrained second end portion of the transfer structure is moved toward the reservoir.

15. The apparatus of claim 8, wherein the force exerted by the actuator is a first force, the apparatus further comprising:
a spring coupled to the transfer structure, a first end portion of the spring being slidably disposed within a channel defined by the housing, the spring configured to exert a second force onto the transfer structure such that the second end portion of the transfer structure is moved toward the reservoir.

16. An apparatus, comprising:
a housing removably couplable to a user;
a reservoir configured to contain a fluid and disposed within the housing;
an actuator having a constrained first end portion and an unconstrained second end portion;
a transfer structure disposed between the actuator and the reservoir,
the actuator being configured so that when actuated, a first force is exerted by the actuator onto the transfer structure; and
a spring coupled to the transfer structure, the spring configured to exert a second force onto the transfer structure, the first force and the second force collectively configured to cause the transfer structure to exert a force on the reservoir such that fluid within the reservoir is communicated out of the reservoir.

17. The apparatus of claim 16, wherein the actuator is an electrochemical actuator.

18. The apparatus of claim 16, wherein a first end portion of the spring is coupled to a roller member configured to slidably move within a channel defined by the housing, the roller member configured to exert the second force onto the transfer structure.

19. The apparatus of claim 16, wherein a first end portion of the spring is coupled to a drive wedge configured to slidably move relative to the transfer structure such that a roller member coupled to the drive wedge exerts the second force on the transfer structure.

20. The apparatus of claim 16, wherein the spring is a compression spring.

21. The apparatus of claim 16, wherein the spring is an extension spring.

22. The apparatus of claim 16, wherein the actuator has a medial portion between the first end portion and the second end portion, the actuator being configured so that when actuated, the medial portion of the actuator bends and imparts the first force on the transfer structure.
